SEQUENCE LISTING (12 sequences)

SEQ ID NO: 1 - DNA, 21 bp, synthetic construct
  source 1..21: mol_type = other DNA; note = Primer PEDV-F
gcacttattggcaggctttgt

SEQ ID NO: 2 - DNA, 25 bp, synthetic construct
  source 1..25: mol_type = other DNA; note = Primer PEDV-R
ccattgagaaaagaaagtgtcgtag

SEQ ID NO: 3 - DNA, 24 bp, synthetic construct
  source 1..24: mol_type = other DNA; note = Primer PDCoV-F
agctcccaagcggactttacccaa

SEQ ID NO: 4 - DNA, 24 bp, synthetic construct
  source 1..24: mol_type = other DNA; note = Primer PDCoV-R
agccatacccgtcttctcagtgtc

SEQ ID NO: 5 - DNA, 21 bp, synthetic construct
  source 1..21: mol_type = other DNA; note = Primer PRV-F
ggtggaccggctgctgaacga

SEQ ID NO: 6 - DNA, 21 bp, synthetic construct
  source 1..21: mol_type = other DNA; note = Primer PRV-R
gctgctggtagaacggcgtca

SEQ ID NO: 7 - DNA, 20 bp, synthetic construct
  source 1..20: mol_type = other DNA; note = Primer PCV-F
ttgtaatgaaggtcccagcc

SEQ ID NO: 8 - DNA, 20 bp, synthetic construct
  source 1..20: mol_type = other DNA; note = Primer PCV-R
gaagtaacgggagtggtagg

SEQ ID NO: 9 - DNA, 20 bp, synthetic construct
  source 1..20: mol_type = other DNA; note = Primer PoRV-F
aactgtaaaaagctaggacc

SEQ ID NO: 10 - DNA, 18 bp, synthetic construct
  source 1..18: mol_type = other DNA; note = Primer PoRV-R
tttcggtctgcggcactg

SEQ ID NO: 11 - DNA, 18 bp, synthetic construct
  source 1..18: mol_type = other DNA; note = Primer PRRSV-F
ctccactacggtcaacgg

SEQ ID NO: 12 - DNA, 18 bp, synthetic construct
  source 1..18: mol_type = other DNA; note = Primer PRRSV-R
acaaggtttaccactccc

(19) United States
(12) Patent Application Publication
(10) Pub. No.: US 2026/0021136 A1
(43) Pub. Date: Jan. 22, 2026

(54) ALKALINE MINERAL COMPOSITION, METHOD OF PREPARATION, AND USE THEREOF

(71) Applicant: NORTHEAST AGRICULTURAL UNIVERSITY, Harbin, Heilongjiang (CN)

(72) Inventors: Jinlong LI, Harbin (CN); Xin YAO, Harbin (CN); Xuenan LI, Harbin (CN); Yi ZHAO, Harbin (CN); Jian CHEN, Harbin (CN); Xiaowei LI, Harbin (CN); Yixi TANG, Harbin (CN)

(73) Assignee: NORTHEAST AGRICULTURAL UNIVERSITY, Harbin, Heilongjiang (CN)

(21) Appl. No.: 18/884,729

(22) Filed: Sep. 13, 2024

(30) Foreign Application Priority Data
Jul. 16, 2024 (CN) 202410956062.3

(51) Int. Cl. A61K 33/30; A61K 9/06; A61K 33/00; A61K 33/04; A61K 33/14; A61K 33/18; A61K 33/24; A61K 33/244; A61K 33/26; A61K 33/38; A61K 47/36; A61P 31/12

(52) U.S. Cl. CPC A61K 33/30; A61K 9/06; A61K 33/00; A61K 33/04; A61K 33/14; A61K 33/18; A61K 33/24; A61K 33/244; A61K 33/26; A61K 33/38; A61K 47/36; A61P 31/12

(57) ABSTRACT

The present disclosure relates to the field of livestock farming and specifically pertains to an alkaline mineral composition, a method of preparation, and use thereof. The alkaline mineral composition includes a metal ion complex, a mineral ion complex, an alkaline soluble rare earth salt, and a composite gel as active ingredients, formulated in specific mass ratios. It can be used as a feed additive, added to drinking water, or directly fed to animals. The components of the alkaline mineral composition in the present disclosure synergistically enhance efficacy, collectively inhibiting replication and transmission of viruses including coronaviruses, circoviruses, rotaviruses, arteriviruses, and herpesviruses in livestock. It enhances the body's toxin-filtering capacity, strengthens host cell antiviral and immune capabilities, and helps the host resist the damage caused by viruses to the body, effectively improving the viral infection in livestock. It can serve as a safe and effective alternative to antiviral vaccines.

### CROSS-REFERENCE TO RELATED APPLICATION

This patent application claims the benefit and priority of Chinese Patent Application No. 2024109560623 filed with the China National Intellectual Property Administration on Jul. 16, 2024, the disclosure of which is incorporated by reference herein in its entirety as part of the present application.

### REFERENCE TO SEQUENCE LISTING

A computer readable XML file entitled “GWP20240705130_seqlist”, which was created on Jul. 26, 2024, with a file size of about 13,537 bytes, contains the sequence listing for this application, has been filed with this application, and is hereby incorporated by reference in its entirety.

### TECHNICAL FIELD

The present disclosure relates to the field of livestock farming technology, in particular to an alkaline mineral composition, method of preparation, and use thereof.

### BACKGROUND

In recent years, with the rapid development of the livestock industry, the supply of livestock products has increased significantly, driving economic growth and improving people's living standards. However, with the expansion of farming scale and intensification, the threat of viral infectious diseases in livestock has become increasingly serious. These diseases not only severely impact the health and productivity of livestock but also result in significant economic losses for farmers and the industry as a whole.

Currently, the main methods for preventing and controlling viral infectious diseases in livestock include immunization through vaccines, the use of antiviral drugs, and antiviral herbal medicines. Vaccines are the most commonly used method for combating viruses in livestock, but they face several challenges. For example, many viruses have multiple serotypes and mutate rapidly, making existing vaccines often ineffective in providing protection, leading to vaccination failures. Some vaccines, although effective in providing immunity, may pose potential safety risks. Antiviral drugs such as ribavirin, amantadine, and lamivudine are effective but can be toxic to host cells, leading to drug resistance with prolonged use, and affecting clinical outcomes. These drugs have already been banned for use in domestic animals. The use of antibiotics can also lead to drug-resistant pathogens and residual presence in the body, affecting the immune systems of humans and animals through the food chain, disrupting intestinal microbial balance, and increasing the risk of cross-infections. As an emerging method for prevention and treatment, antiviral herbal medicines have played a role in combating viral infectious diseases in livestock. However, their use in feed and drinking water can result in residual peculiar odor, which affects the export of meat products and limits their international application. Furthermore, the effective components and mechanisms of action of herbal medicines are not fully understood, and it is difficult to control the concentration of active components, resulting in unstable antiviral effects in different environmental conditions and impacting the reliability of treatment outcomes.

In conclusion, although existing technologies and drugs have to some extent controlled and alleviated viral infections in livestock, there are still many shortcomings and limitations. There is currently no ideal, broad-spectrum antiviral drug with strong efficacy. The long-term health and development of the livestock industry urgently require the development of a more effective means of prevention and control to address the challenges of viral variations and new viruses in livestock farming, ensuring the health of livestock and the safety of livestock products.

### SUMMARY

In order to overcome the problems of the prior art mentioned above, the present disclosure aims to provide an alkaline mineral composition, a method of preparation, and use thereof. The alkaline mineral composition of the present disclosure has strong antiviral effects and broad-spectrum antiviral properties, making it an effective alternative to antiviral vaccines.

The alkaline mineral composition provided in the present disclosure includes the following components in parts by weight: 60-70 parts of metal ion complex, 25-30 parts of mineral ion complex, 1-9.5 parts of alkaline soluble rare earth salt, and 0.5-4 parts of composite gel.

In some embodiments, the metal ion complex includes a sodium compound, a potassium compound, a zinc compound, and an iron compound; sodium ions, potassium ions, zinc ions, and iron ions in the sodium compound, the potassium compound, the zinc compound, and the iron compound have a mass ratio of (16.48-19.02):(41.21-43.18):(0.23-0.25):(2.06-7.93); the sodium compound includes sodium chloride, sodium carbonate, sodium selenite, and sodium metasilicate; the potassium compound includes potassium chloride, potassium iodide, and potassium carbonate; the zinc compound includes zinc gluconate; the iron compound includes ferrous sulfate, ferrous lactate, and ferrous gluconate.

In some embodiments, the mineral ion complex includes a selenium compound, an iodine compound, and a silicon compound; selenium ions, iodine ions, and silicon ions in the selenium compound, the iodine compound, and the silicon compound have a mass ratio of (1.14-4.67):(18.66-25):(1.14-4.67); the selenium compound includes sodium selenite; the iodine compound includes potassium iodide; the silicon compound includes sodium metasilicate.

In some embodiments, the alkaline soluble rare earth salt includes digermanate and lanthanum carbonate; the digermanate and the lanthanum carbonate has a mass ratio of (0.87-3.8):(0.13-5.7).

In some embodiments, the composite gel includes sodium alginate and nano silver; the sodium alginate and the nano silver have a mass ratio of (0.48-3):(0.02-1).

A method for preparing the alkaline mineral composition provided in the present disclosure includes the following steps: dissolving the metal ion complex, the mineral ion complex, and the alkaline soluble rare earth salt in sequence in the composite gel, and performing ionization to obtain the alkaline mineral composition.

In some embodiments, the ionization is performed at a temperature of 25-35° C. and at an ionization potential of −25 to 25 mV for 1-2 days.

The present disclosure also provides an antiviral preparation, including the alkaline mineral composition as described above or an alkaline mineral composition prepared by the method.

The present disclosure further provides use of the alkaline mineral composition as described above or prepared by the method or the antiviral preparation in preparation of a drug for preventing and/or treating infections in livestock caused by a virus.

In some embodiments, the virus includes a coronavirus, a circovirus, a rotavirus, an arterivirus, and a herpesvirus.

### Beneficial Effects

The present disclosure provides an alkaline mineral composition, a method of preparation, and use thereof. The alkaline mineral composition includes the following raw materials in mass ratios: 60-70 parts of metal ion complex, 25-30 parts of mineral ion complex, 1-9.5 parts of alkaline soluble rare earth salt, and 0.5-4 parts of composite gel. In the composition of the present disclosure, the metal ion complex promotes blood circulation, maintains internal stability such as water balance and energy metabolism, enhances antioxidant activity, improves immune system function, regulates metabolism and body development, improves intestinal morphology, and helps enhance host immunity. The mineral ion complex reduces cell damage caused by free radicals, enhances immune system function, regulates host lipid metabolism, lowers cholesterol, promotes bile acid synthesis, inhibits pathogen transmission, provides energy, and effectively alleviates immune deficiencies due to inadequate nutrient intake. The alkaline soluble rare earth salt promotes blood circulation, lowers serum phosphate and calcium phosphate levels, and enhances the body's toxin filtration capacity. The composite gel inherently strengthens the intestinal barrier, helping the host resist viral damage to the body.

In the alkaline mineral composition of the present disclosure, there is a synergistic effect among the components, significantly enhancing the regulation of lipid metabolism in the body. As viruses complete multiple steps of their lifecycle through complex interactions with host cell lipid metabolism, the alkaline mineral composition of the present disclosure inhibits viral replication and transmission by manipulating host lipid metabolism, thereby achieving targeted antiviral treatment. Additionally, the combination of components synergistically enhances the body's toxin filtration capacity, improving the efficiency of virus clearance. Furthermore, the use of the combined components may further enhance the protective effect of the intestinal barrier, balance immune function through intestinal barrier regulation, enhance host cell antiviral and immune capabilities, and effectively inhibit virus invasion and infection. As a base for the alkaline mineral composition, the composite gel may combine the metal ion complex, the mineral ion complex, and the alkaline soluble rare earth salt, ensuring product quality stability and achieving a broad-spectrum antiviral effect to a maximum extent. It exhibits excellent inhibitory effects on various viruses, including coronaviruses, circoviruses, rotaviruses, arteriviruses, and herpesviruses, serving as an alternative for broad-spectrum antiviral vaccines.

### BRIEF DESCRIPTION OF THE DRAWINGS

To better illustrate the specific embodiments of the present disclosure or the technical solutions in the prior art, a brief introduction of the drawings required in the examples will be provided below. It is evident that the descriptions of the drawings below are just some embodiments of the present disclosure, and ordinary skilled artisans in the art can derive other drawings without an inventive step based on these drawings.

FIG. 1 is a schematic diagram of the animal experimental process in Application Example 1;

FIG. 2 shows bar plots of the viral load in animal rectal swab samples in Application Example 1;

FIG. 3A-FIG. 3D illustrate results of transcriptomics and metabolomics analysis in Application Example 2, where FIG. 3A shows changes in gene expression levels in animals, FIG. 3B shows enrichment results of differentially expressed genes (DEGs), FIG. 3C shows changes in metabolic expression levels in animals, and FIG. 3D shows classification of differentially expressed metabolites (DEMs);

FIG. 4 illustrates detection results of serum lipid-related biochemical indicators in Application Example 2;

FIG. 5A-FIG. 5B illustrate images showing colon periodic acid-Schiff (PAS) staining and colon oil red O staining in Application Example 2, where FIG. 5A shows images of colon PAS staining, with the scale bar being 100 μm for the upper row images and 20 μm for the lower row images, and FIG. 5B shows images of colon oil red O staining, with the scale bar being 20 μm for the upper row images and 100 μm for the middle row images;

FIG. 6A-FIG. 6B illustrate images of colon hematoxylin and eosin (H&E) staining and scanning electron microscopy (SEM) in Application Example 3, where FIG. 6A shows images of H&E staining, with the scale bar being 100 μm for the upper row images and 20 μm for the lower row images, and FIG. 6B shows images of SEM, with the scale bar being 1 μm for the upper row images (10.0k×magnification) and 0.5 μm for the lower row images (30.0 k×magnification);

FIG. 7 illustrates a schematic diagram of the cell experiment process in Application Example 4;

FIG. 8 illustrates results of the viral replication ratio in the cell culture supernatant in Application Example 4;

FIG. 9 illustrates immunofluorescence images of viral infection in cells in Application Example 4;

FIG. 10 illustrates fluorescence images of Nile red staining in cells in Application Example 5;

FIG. 11 illustrates bar plots showing bacterial density in Application Example 6;

FIG. 12 is a bar plot showing antiviral effects of the alkaline mineral composition and its components in Application Example 7.

### DETAILED DESCRIPTION OF THE EMBODIMENTS

The present disclosure provides an alkaline mineral composition, including the following components in parts by weight: 60-70 parts of metal ion complex, 25-30 parts of mineral ion complex, 1-9.5 parts of alkaline soluble rare earth salt, and 0.5-4 parts of composite gel.

In the present disclosure, the alkaline mineral composition preferably includes the following components in parts by weight: 60-70 parts of metal ion complex, 25-30 parts of mineral ion complex, 1-9.5 parts of alkaline soluble rare earth salt, and 0.5-4 parts of composite gel. The synergistic effect of the components in this composition can jointly inhibit the replication and transmission of viruses in livestock, enhance the body's toxin filtration capacity, improve the host cell's antiviral and immune capabilities, and help the host resist the damage caused by viruses to the body.

In the present disclosure, by parts by weight, the alkaline mineral composition includes 60-70 parts of metal ion complex, preferably 65 parts. In the present disclosure, the metal ion complex can enhance blood circulation, maintain internal stability such as water balance and energy metabolism, improve antioxidant activity, promote the enhancement of immune system function, regulate metabolism and body development, and help improve host immunity.

In the present disclosure, the metal ion complex preferably includes a sodium compound, a potassium compound, a zinc compound, and an iron compound. Sodium ions, potassium ions, zinc ions, and iron ions in the sodium compound, the potassium compound, the zinc compound, and the iron compound preferably have a mass ratio of (16.48-19.02):(41.21-43.18):(0.23-0.25):(2.06-7.93), more preferably 17.27:43.18:0.23:4.32.

In the present disclosure, the sodium compound preferably includes one or more of sodium chloride, sodium carbonate, sodium selenite, and sodium metasilicate, more preferably sodium chloride, sodium selenite, and sodium metasilicate. The specific types of sodium compound and the mass ratios of different types are not specifically limited in the present disclosure, as long as the final sodium ion content is met. The specific sources of sodium compound in the present disclosure are not specifically limited, and the compound can be purchased as food-grade by ordinary skilled persons in the art. In the present disclosure, the potassium compound preferably includes one or more of potassium chloride, potassium iodide, and potassium carbonate, more preferably potassium chloride and potassium iodide. The specific types of potassium compound and the mass ratios of different types are not specifically limited in the present disclosure, as long as the final potassium ion content is met. The specific sources of the potassium compound in the present disclosure are not specifically limited, and the compound can be purchased as food-grade by ordinary skilled persons in the art. In the present disclosure, the zinc compound preferably includes zinc gluconate. The specific types of the zinc compound and the mass ratios of different types are not specifically limited in the present disclosure, as long as the final zinc ion content is met. The specific sources of the zinc compound in the present disclosure are not specifically limited, and the compound can be purchased as food-grade by ordinary skilled persons in the art. In the present disclosure, the iron compound preferably includes one or more of ferrous sulfate, ferrous lactate, and ferrous gluconate, more preferably ferrous gluconate. The specific types of the iron compound and the mass ratios of different types are not specifically limited in the present disclosure, as long as the final iron ion content is met. The specific sources of the iron compound in the present disclosure are not specifically limited, and the compound can be purchased as food-grade by ordinary skilled persons in the art.

The present disclosure does not specify a particular preparation process for the metal ion complex, and the components are simply mixed uniformly. In the present disclosure, the metal ion complex contains multiple ions, each with different properties. Sodium ions help maintain fluid balance in the body. Potassium ions are crucial for balancing the electrical potential both inside and outside of cells. When sodium and potassium ions combine with water molecules, they form hydrated ions, which retain water and prevent its loss. This process is important in alleviating dehydration caused by diarrhea. Additionally, these ions are essential for nerve conduction and muscle contraction. Zinc ions are components of various enzymes, and are involved in cell differentiation, immune function, growth and development, and DNA synthesis, thereby enhancing the body's immunity. Iron ions participate in hemoglobin synthesis and oxygen transport, helping maintain the body's homeostasis.

In the present disclosure, by parts by weight, the alkaline mineral composition includes 25-30 parts of mineral ion complex, preferably 28 parts. In the present disclosure, the mineral ion complex can reduce cell damage caused by free radicals, enhance immune system function, regulate host lipid metabolism, lower cholesterol levels, promote bile acid synthesis, inhibit pathogen transmission, provide energy, and effectively alleviate immune deficiencies due to inadequate nutrient intake.

In the present disclosure, the mineral ion complex preferably includes a selenium compound, an iodine compound, and a silicon compound. Selenium ions, iodine ions, and silicon ions in selenium compound, the iodine compound, and the silicon compound preferably have a mass ratio of (1.14-4.67):(18.66-25):(1.14-4.67), more preferably 4.67:18.66:4.67. In the present disclosure, the selenium compound preferably includes sodium selenite. The specific types of selenium compound and the mass ratios of different types are not specifically limited in the present disclosure, as long as the final selenium ion content is met. The specific sources of selenium compound in the present disclosure are not specifically limited, and the compound can be purchased as food-grade by ordinary skilled persons in the art. In the present disclosure, the iodine compound preferably includes potassium iodide. The specific types of the iodine compound and the mass ratios of different types are not specifically limited in the present disclosure, as long as the final iodine ion content is met. The specific sources of iodine compound in the present disclosure are not specifically limited, and the compound can be purchased as food-grade by ordinary skilled persons in the art. In the present disclosure, the silicon compound preferably includes sodium metasilicate. The specific types of the silicon compound and the mass ratios of different types are not specifically limited in the present disclosure, as long as the final silicon ion content is met. The specific sources of the silicon compound in the present disclosure are not specifically limited, and the compound can be purchased as food-grade by ordinary skilled persons in the art.

In the present disclosure, there is no specific limitation on the preparation of the mineral ion complex, and the components are simply mixed uniformly. In the present disclosure, the mineral ion complex contains multiple ions, each with different properties: selenium ions have both growth-promoting and antioxidant effects, playing an important role in regulating intestinal metabolism and development, as well as alleviating oxidative stress; iodine ions regulate the synthesis and metabolism of thyroid hormones, maintain host nutrition by regulating lipid metabolism synthesis mediated by endocrine hormones; silicon ions participate in collagen synthesis, maintain bone health and strength, and also maintain cell membrane barriers to resist viral invasion.

In the present disclosure, by parts by weight, the alkaline mineral composition includes 1-9.5 parts of alkaline soluble rare earth salt, preferably 5 parts. In the present disclosure, the alkaline soluble rare earth salt can promote blood circulation, lower serum phosphate and calcium phosphate levels, and enhance the body's toxin filtration capacity. In the present disclosure, the alkaline soluble rare earth salt preferably includes digermanate and lanthanum carbonate, with a mass ratio of 3.75:1.25. Both the digermanate and the lanthanum carbonate in the present disclosure are preferably in soluble salt form. There is no specific limitation on the source of digermanate and lanthanum carbonate in the present disclosure, and the compounds can be purchased as food-grade by ordinary skilled persons in the art. There is no specific limitation on the preparation of the alkaline soluble rare earth salt in the present disclosure, and the components are simply mixed uniformly. In the present disclosure, the lanthanum carbonate can lower serum phosphate and calcium phosphate levels, regulate plasma proteins such as acid glycoproteins, serum albumin, and transferrin, and enhance the body's toxin filtration capacity; digermanate can promote blood circulation, lower blood lipids, exhibit antioxidant properties, and enhance immunity.

In the present disclosure, by parts by weight, the alkaline mineral composition includes 0.5-4 parts, preferably 2 parts of composite gel. In the present disclosure, the composite gel inherently strengthens the intestinal barrier, helping the host resist the damage caused by viruses to the body.

In the present disclosure, the composite gel preferably includes sodium alginate and nano silver, with a mass ratio of the sodium alginate to the nano silver preferably ranging from (0.48-3):(0.02-1), more preferably 1.5:0.5. In the present disclosure, the nano silver is preferably in the form of nanoparticles. There is no specific limitation on the source of nano silver in the present disclosure, and nano-grade silver commonly purchased by skilled persons in the art can be used. There is no specific limitation on the source of the sodium alginate in the present disclosure, and food-grade sodium alginate commonly purchased by skilled persons in the art can be used.

In the present disclosure, a method for preparing the composite gel preferably includes the following steps: mixing the sodium alginate and the nano silver, performing a first dissolution to obtain a first dissolved product; mixing the first dissolved product with water to perform a second dissolution, conducting stirring in the dark, and allowing to stand to obtain the composite gel. In the present disclosure, the first dissolved product and water preferably have a mass-volume ratio of (0.5-4) g:1 L; the first dissolution is preferably conducted at a temperature of 25-35° C., more preferably 27° C., preferably by standing for 1-2 days, more preferably 1 day; the second dissolution is preferably conducted at a temperature of 4-12° C., more preferably 4° C., by stirring for 1-6 hours, more preferably 3 hours, followed by standing for 1-2 days, more preferably 1 day. In a specific embodiment of the present disclosure, the method for preparing the composite gel includes uniformly mixing the sodium alginate and the nano silver, adding water to dissolve in the dark at 27° C. for 1 day, stirring in the dark at 4° C. for 3 hours, and standing for 1 day to prepare the composite gel. In another specific embodiment of the present disclosure, the method for preparing the composite gel includes uniformly mixing the sodium alginate and the nano silver, adding water to dissolve in the dark at 35° C. for 2 days, stirring in the dark at 12° C. for 6 hours, and standing for 1 day to prepare the composite gel. In another specific embodiment of the present disclosure, the method for preparing the composite gel includes uniformly mixing the sodium alginate and the nano silver, adding water to dissolve in the dark at 25° C. for 1.5 days, stirring in the dark at 8° C. for 1 hour, and standing for 1.5 days to prepare the composite gel. In the present disclosure, the sodium alginate alginate, as a gel, possesses excellent moisturizing properties, can form emulsions between water and oil, aid in the mixing and stabilization of immiscible components, and enhance the texture and stability of products. The nano silver has superior antibacterial properties and anti-inflammatory characteristics, which can alleviate inflammatory responses and promote wound healing.

The present disclosure further provides a method for preparing the alkaline mineral composition, including the following steps:

dissolving the metal ion complex, the mineral ion complex, and the alkaline soluble rare earth salt sequentially in the composite gel, and performing ionization to obtain the alkaline mineral composition. In the present disclosure, the ionization is preferably performed at a temperature of 25-35° C., more preferably 37° C. and an ionization potential of −25 to 25 mV, for 1-2 days, more preferably 2 days. In a specific embodiment of the present disclosure, the method for preparing the alkaline mineral composition includes sequentially dissolving the metal ion complex, the mineral ion complex, and the alkaline soluble rare earth salt in the composite gel, performing ionization under a nitrogen atmosphere, with an ionization temperature of 27° C., an ionization potential of −25 to 25 mV, and an ionization time of 2 days. In another specific embodiment of the present disclosure, the method for preparing the alkaline mineral composition includes sequentially dissolving the metal ion complex, the mineral ion complex, and alkaline soluble rare earth salt in the composite gel, performing ionization under a nitrogen atmosphere, with an ionization temperature of 25° C., an ionization potential of −25 to 25 mV, and an ionization time of 1 day. In yet another specific embodiment of the present disclosure, the method for preparing the alkaline mineral composition includes sequentially dissolving the metal ion complex, the mineral ion complex, and the alkaline soluble rare earth salt in the composite gel, performing ionization under a nitrogen atmosphere, with an ionization temperature of 30° C., an ionization potential of −25 to 25 mV, and an ionization time of 1.5 days. In the present disclosure, sequentially dissolving the metal ion complex, the mineral ion complex, and the alkaline soluble rare earth salt in the composite gel for ionization can lead to specific ionization and recombination among the components in the alkaline mineral composition, further enhancing the product stability of the alkaline mineral composition.

The present disclosure also provides an antiviral preparation, including the alkaline mineral composition as described in the above technical solutions or the alkaline mineral composition prepared by the method of preparation. The virus preferably includes a coronavirus, a circovirus, a rotavirus, an arterivirus, and a herpesvirus. The coronavirus preferably includes porcine epidemic diarrhea virus (PEDV) and porcine deltacoronavirus (PDCoV). The circovirus preferably includes porcine circovirus (PCV). The rotavirus preferably includes porcine rotavirus (PORV). The arterivirus preferably includes porcine reproductive and respiratory syndrome virus (PRRSV). The herpesvirus preferably includes PRV. The preparation preferably also includes a pharmaceutically acceptable excipient. The type and amount of the excipient in the preparation are not specifically limited and any pharmaceutically acceptable excipients can be used. The dosage form of the preparation is not specifically limited and any pharmaceutically acceptable dosage form, such as tablets, oral liquids, capsules, granules, powders, pills, etc. can be used. In a specific embodiment of the present disclosure, the dosage form of the preparation is a liquid formulation, and the solvent of the preparation is preferably H₂O. The method for administering the liquid formulation includes diluting the preparation with water, feeding piglets after dilution at a ratio of 1000 times, with a feeding amount of 100 mL/kg/d. In the present disclosure, the concentration of the drug can be optimized and adjusted depending on livestock species, animal age, and health conditions of the animals. In the present disclosure, the alkaline mineral composition can be tailored for symptomatic treatment according to the actual pathological course of livestock. Specifically, it can be administered according to the clinical experience of veterinarians, depending on, e.g., the severity of diarrhea, water content and color of the feces, and the ability of livestock to walk normally. The combination of components in the composite preparation of the present disclosure is scientifically reasonable, maximizing the effectiveness of each component.

The present disclosure also provides use of the alkaline mineral composition or the alkaline mineral composition prepared by the preparation method in the preparation of a drug for preventing and/or treating viral infections in livestock. In the present disclosure, the livestock preferably includes a pig, cattle, or sheep, where the pig is preferably a newborn piglet.

In the present disclosure, the use preferably includes use of the alkaline mineral composition in the preparation of a drug that inhibits replication and transmission of a virus, enhances toxin filtration capacity of livestock, improves antiviral and immune capabilities of livestock, helps livestock resist damage caused by the virus to the body, or improves viral infection of livestock.

In the present disclosure, the virus preferably includes one or more of a coronavirus, a circovirus, a rotavirus, an arterivirus, and a herpesvirus.

To further illustrate the present disclosure, a detailed description of the alkaline mineral composition, the method of preparation, and use thereof provided in the present disclosure will be given in conjunction with the drawings and examples. However, this should not be construed as limiting the scope of the present disclosure.

### Example 1. Preparation of Alkaline Mineral Metal Composition A

### 1.1 Preparation of Raw Materials

Sixty-five grams of metal ions from metal ion complex (containing a total of 17.27 g of sodium ions from sodium compounds, a total of 43.18 g of potassium ions from potassium compounds, a total of 0.23 g of zinc ions from zinc compounds, and a total of 4.32 g of iron ions from iron compounds), 28 g of mineral ions from mineral ion complex (containing a total of 4.67 g of selenium ions from selenium compounds, a total of 18.66 g of iodine ions from iodine compounds, and a total of 4.67 g of silicon ions from silicon compounds), 5 g of alkaline soluble rare earth salt (3.75 g of digermanate and 1.25 g of lanthanum carbonate), and 2 g of composite gel (1.5 g of sodium alginate and 0.5 g of nano silver) were prepared.

In the metal ion complex, 17.27 g of sodium ions were derived from sodium compounds, including 17.62 g of sodium chloride, 11.18 g of sodium selenite, and 20.29 g of sodium metasilicate; 43.18 g of potassium ions were derived from potassium compounds, including 71.38 g of potassium chloride and 24.41 g of potassium iodide; 0.23 g of zinc ions were derived from zinc compounds, including 1.6 g of zinc gluconate; and 4.32 g of iron ions were derived from iron compounds, including 37.2 g of ferrous gluconate.

In the mineral ion complex, 18.66 g of iodine ions were derived from iodine compounds, including 24.41 g of potassium iodide; 4.67 g of selenium ions were derived from selenium compounds, including 11.18 g of sodium selenite; and 4.67 g of silicon ions were derived from silicon compounds, including 20.29 g of sodium metasilicate.

### 1.2 Preparation of the Alkaline Mineral Composition

1) Sodium alginate (1.5 g) and nano silver (0.5 g) were uniformly mixed and dissolved in 1 L of water at 27° C. in the dark for 1 day. Subsequently, the mixture was stirred in the dark at 4° C. for 3 hours and allowed to stand for 1 day to obtain the composite gel. 2) Sodium compounds with a total sodium ion content of 17.27 g, potassium compounds with a total potassium ion content of 43.18 g, zinc compounds with a total zinc ion content of 0.23 g, and iron compounds with a total iron ion content of 4.32 g were uniformly mixed to prepare the metal ion complex. 3) Selenium compounds with a total selenium ion content of 4.67 g, iodine compounds with a total iodine ion content of 18.66 g, and silicon compounds with a total silicon ion content of 4.67 g were uniformly mixed to prepare the mineral ion complex. 4) Digermanate (3.75 g) and lanthanum carbonate (1.25 g) were uniformly mixed to prepare the alkaline soluble rare earth salt. 5) The metal ion complex obtained in step 2), the mineral ion complex obtained in step 3), and the alkaline soluble rare earth salt obtained in step 4) were sequentially added to the composite gel obtained in step 1). The mixture was uniformly mixed and ionized in a nitrogen atmosphere at 27° C. with a potential of −25 to 25 mV for 2 days to obtain the alkaline mineral composition, which was stored at room temperature for future use.

### Example 2. Preparation of Alkaline Mineral Metal Composition B

### 1.1 Preparation of Raw Materials

Sixty grams of metal ions from metal ion complex (containing a total of 16.48 g of sodium ions from sodium compounds, a total of 41.21 g of potassium ions from potassium compounds, a total of 0.25 g of zinc ions from zinc compounds, and a total of 2.06 g of iron ions from iron compounds), 30 g of mineral ion complex (containing a total of 2.5 g of selenium ions from selenium compounds, a total of 25 g of iodine ions from iodine compounds, and a total of 2.5 g of silicon compounds), 9.5 g of alkaline soluble rare earth salt (3.8 g of digermanate and 5.7 g of lanthanum carbonate), and 0.5 g of composite gel (0.48 g of sodium alginate and 0.02 g of nano silver) were prepared.

In the metal ion complex, 16.48 g of sodium ions were derived from sodium compounds, including 27.78 g of sodium chloride, 5.98 g of sodium selenite, and 10.86 g of sodium metasilicate; 41.21 g of potassium ions were derived from potassium compounds, including 63.9 g of potassium chloride and 32.7 g of potassium iodide; 0.25 g of zinc ions were derived from zinc compounds, including 1.74 g of zinc gluconate; 2.06 g of iron ions were derived from iron compounds, including 17.74 g of ferrous gluconate.

In the mineral ion complex, 25 g of iodine ions were derived from iodine compounds, including 32.7 g of potassium iodide; 2.5 g of selenium ions were derived from selenium compounds, including 5.98 g of sodium selenite; and 2.5 g of silicon ions were derived from silicon compounds, including 10.86 g of sodium metasilicate.

### 1.2 Preparation of Alkaline Mineral Metal Composition

1) Sodium alginate (0.48 g) and nano silver (0.02 g) were uniformly mixed and dissolved in 1 L of water at 35° C. in the dark for 2 days. Subsequently, the mixture was stirred in the dark at 12° C. for 6 hours and allowed to stand for 1 day to obtain the composite gel. 2) The sodium compounds containing a total of 16.48 g of sodium ions, the potassium compounds containing a total of 41.21 g of potassium ions, the zinc compounds containing a total of 0.25 g of zinc ions, and the iron compounds containing a total of 2.06 g of iron ions were uniformly mixed to prepare the metal ion complex. 3) The selenium compounds containing a total of 2.5 g of selenium ions, the iodine compounds containing a total of 25 g of iodine ions, and the silicon compounds containing a total of 2.5 g of silicon ions were uniformly mixed to prepare the mineral ion complex. 4) Digermanate (3.8 g) and lanthanum carbonate (5.7 g) were uniformly mixed to prepare the alkaline soluble rare earth salt. 5) The metal ion complex obtained in step 2, the mineral ion complex obtained in step 3, and the alkaline soluble rare earth salt obtained in step 4 were sequentially added to the composite gel obtained in step 1). The mixture was uniformly mixed and ionized in a nitrogen atmosphere at 25° C. with a potential of −25 to 25 mV for 1 day to obtain the alkaline mineral composition, which was stored at room temperature for future use.

### Example 3. Preparation of Alkaline Mineral Metal Composition C

### 1.1 Preparation of Raw Materials

Seventy grams of metal ions from metal ion complex (including a total of 19.02 g of sodium ions from sodium compounds, a total of 42.81 g of potassium ions from potassium compounds, a total of 0.24 g of zinc ions from zinc compounds, and a total of 7.93 g of iron ions from iron compounds), 25 g of mineral ions from mineral ion complex (containing a total of 1.14 g of selenium ions from selenium compounds, a total of 22.72 g of iodine ions from iodine compounds, and a total of 1.14 g of silicon ions from silicon compounds), 1 g of alkaline soluble rare earth salt (0.87 g of digermanate and 0.13 g of lanthanum carbonate), and 4 g of composite gel (3 g of sodium alginate and 1 g of nano silver) were prepared.

In the metal ion complex, 19.02 g of sodium ions were derived from sodium compounds, including 41.94 g of sodium chloride, 2.73 g of sodium selenite, and 4.95 g of sodium metasilicate; 42.81 g of potassium ions were derived from potassium compounds, including 68.29 g of potassium chloride and 29.72 g of potassium iodide; 0.24 g of zinc ions were derived from zinc compounds, including 1.67 g of zinc gluconate; and 7.93 g of iron ions were derived from iron compounds, including 68.28 g of ferrous gluconate.

In the mineral ion complex, 22.72 g of iodine ions were derived from iodine compounds, including 29.72 g of potassium iodide; 1.14 g of selenium ions were derived from selenium compounds, including 2.73 g of sodium selenite; 1.14 g of silicon ions were derived from silicon compounds, including 4.95 g of sodium metasilicate.

### 1.2 Preparation of Alkaline Mineral Metal Composition

1) Sodium alginate (3 g) and nano silver (1 g) were uniformly mixed and dissolved in 1 L of water at 25° C. in the dark for 1.5 days. Subsequently, the mixture was stirred in the dark at 8° C. for 1 hour and allowed to stand for 1.5 days to obtain the composite gel. 2) The sodium compounds containing a total of 19.02 g of sodium ions, the potassium compounds containing a total of 42.81 g of potassium ions, the zinc compounds containing a total of 0.24 g of zinc ions, and the iron compounds containing a total of 7.93 g of iron ions were uniformly mixed to prepare the metal ion complex. 3) The selenium compounds containing a total of 1.14 g of selenium ions, the iodine compounds containing a total of 22.72 g of iodine ions, and the silicon compounds containing a total of 1.14 g of silicon ions were uniformly mixed to prepare the mineral ion complex. 4) Digermanate (0.87 g) and lanthanum carbonate (0.13 g) were uniformly mixed to prepare the alkaline soluble rare earth salt. 5) The metal ion complex obtained in step 2, the mineral ion complex obtained in step 3, and the alkaline soluble rare earth salt obtained in step 4 were sequentially added to the composite gel obtained in step 1). The mixture was uniformly mixed and ionized in a nitrogen atmosphere at 30° C. with a potential of −25 to 25 mV for 1.5 days to obtain the alkaline mineral composition, which was stored at room temperature for future use.

### Comparative Example 1. Preparation of Single-Component Material A1 Consisting of the Metal Ion Complex

### 1.1 Preparation of Raw Materials

One hundred grams of metal ions from metal ion complex (containing a total of 26.57 g of sodium ions from sodium compounds, a total of 66.44 g of potassium ions from potassium compounds, a total of 0.35 g of zinc ions from zinc compounds, and a total of 6.64 g of iron ions from iron compounds) were prepared.

In the metal ion complex, 26.57 g of sodium ions were derived from sodium compounds, including 67.54 g of sodium chloride; 66.44 g of potassium ions were derived from potassium compounds, including 126.69 g of potassium chloride; 0.35 g of zinc ions were derived from zinc compounds, including 2.44 g of zinc gluconate; and 6.64 g of iron ions were derived from iron compounds, including 57.17 g of ferrous gluconate.

### 1.2 Preparation of the Single-Component Material A1

1) The sodium compounds with a total sodium ion content of 26.57 g, the potassium compounds with a total potassium ion content of 66.44 g, the zinc compounds with a total zinc ion content of 0.35 g, and the iron compounds with a total iron ion content of 6.64 g were uniformly mixed to prepare the metal ion complex. 2) The metal ion complex obtained in step 1 was dissolved in 1 L of water at 27° C. in the dark for 1 day. Subsequently, the mixture was stirred in the dark at 4° C. for 3 hours and allowed to stand for 1 day to obtain the mixture. 3) The mixture was ionized in a nitrogen atmosphere at 27° C. with a potential of −25 to 25 mV for 2 days to obtain the single-component material A1 consisting of the metal ion complex, which was stored at room temperature for future use.

### Comparative Example 2. Preparation of Single-Component Material A2 Consisting of the Alkaline Mineral Complex

### 1.1 Preparation of Raw Materials

One hundred grams of alkaline minerals from the alkaline mineral complex (containing a total of 16.67 g of selenium ions from selenium compounds, a total of 66.66 g of iodine ions from iodine compounds, and a total of 16.67 g of silicon ions from silicon compounds) were prepared.

In the alkaline mineral composite, 66.66 g of iodine ions were derived from iodine compounds, including 87.2 g of potassium iodide; 16.67 g of selenium ions were derived from selenium compounds, including 39.89 g of sodium selenite; 16.67 g of silicon ions were derived from silicon compounds, including 72.44 g of sodium metasilicate.

### 1.2 Preparation of the Single-Component Material A2

1) The selenium compounds with a total selenium ion content of 16.67 g, the iodine compounds with a total iodine ion content of 66.66 g, and the silicon compounds with a total silicon ion content of 16.67 g were uniformly mixed to prepare the alkaline mineral composite. 2) The alkaline mineral composite obtained in step 1 was dissolved in 1 L of water at 27° C. in the dark for 1 day. Subsequently, the mixture was stirred in the dark at 4° C. for 3 hours and allowed to stand for 1 day to obtain the mixture. 3) The mixture was ionized in a nitrogen atmosphere at 27° C. with a potential of −25 to 25 mV for 2 days to obtain the single-component material A2 consisting of the alkaline mineral complex, which was stored at room temperature for future use.

### Comparative Example 3. Preparation of Single-Component Material A3 Consisting of the Alkaline Soluble Rare Earth Salt

### 1.1 Preparation of Raw Materials

One hundred grams of alkaline soluble rare earth salt (containing 75 g of digermanate and 25 g of lanthanum carbonate) were prepared.

### 1.2 Preparation of the Single-Component Material A3

1) 75 g of digermanate and 25 g of lanthanum carbonate were uniformly mixed to prepare the alkaline mineral composite. 2) The alkaline soluble rare earth salt obtained in step 1 was dissolved in 1 L of water at 27° C. in the dark for 1 day. Subsequently, the mixture was stirred in the dark at 4° C. for 3 hours and allowed to stand for 1 day to obtain the mixture. 3) The mixture was ionized in a nitrogen atmosphere at 27° C. with a potential of −25 to 25 mV for 2 days to obtain single-component material A3 consisting of the alkaline soluble rare earth salt, which was stored at room temperature for future use.

### Comparative Example 4. Preparation of Single-Component Material A4 Consisting of the Composite Gel

### 1.1 Preparation of Raw Materials

100 g of composite gel (containing 60 g of sodium alginate and 40 g of nano silver).

### 1.2 Preparation of Single-Component Material A4

1) Sodium alginate (60 g) and nano silver (40 g) were uniformly mixed to prepare the composite gel. 2) The composite gel obtained in step 1 was dissolved in 1 L of water at 27° C. in the dark for 1 day. Subsequently, the mixture was stirred in the dark at 4° C. for 3 hours and allowed to stand for 1 day to obtain the mixture. 3) The mixture was ionized in a nitrogen atmosphere at 27° C. with a potential of −25 to 25 mV for 2 days to obtain single-component material A4 consisting of the composite gel, which was stored at room temperature for future use.

### Comparative Example 5. Preparation of Anti-Viral Composite Formulation D

### 1.1 Preparation of Raw Materials

Twenty-five grams of metal ions from metal ion complex (containing a total of 17 g of sodium ions from sodium compounds, a total of 7.3 g of potassium ions from potassium compounds, a total of 0.05 g of zinc ions from zinc compounds, a total of 0.05 g of germanium ions from germanium compounds, a total of 0.2 g of titanium ions from titanium compounds, a total of 0.1 g of magnesium ions from magnesium compounds, and a total of 0.3 g of manganese ions from manganese compounds), 68 g of composite reducing sugar preparation (9.3 g of reducing sugar, 49.4 g of rice bran oil, and 9.3 g of brown sugar), and 7 g of composite probiotics (yeast, lactobacillus, bacillus, and photosynthetic bacteria in a mass ratio of 1:2:1:0.4, with yeast activity of 2 billion clony-forming units (CFU)/g, lactobacillus activity of 0.3 billion CFU/g, bacillus activity of 1 billion CFU/g, and photosynthetic bacteria activity of 0.3 billion CFU/g).

In the metal ion complex, 17 g of sodium ions were derived from a sodium compound, which was 62.08 g of sodium bicarbonate; 7.3 g of potassium ions were derived from a potassium compound, which was 12.9 g of potassium carbonate; 0.05 g of zinc ions were derived from a zinc compound, which was 0.35 g of zinc gluconate; 0.05 g of germanium ions were derived from a germanium compound, which was 0.12 g of organic germanium; 0.2 g of titanium ions were derived from a titanium compound, which was 0.33 g of titanium dioxide; 0.1 g of magnesium ions were derived from magnesium compound, which was 1.73 g of magnesium gluconate; 0.3 g of manganese ions were derived from a manganese compound, which was 0.68 g of manganese chloride. The composite reducing sugar preparation consisted of equal parts of glucose and fructose.

### 1.2 Preparation of the Anti-Viral Composite Formulation D

1) The sodium compounds containing a total of 17 g of sodium ions, the potassium compounds containing a total of 7.3 g of potassium ions, the zinc compounds containing a total of 0.05 g of zinc ions, the germanium compounds containing a total of 0.05 g of germanium ions, the titanium compounds containing a total of 0.2 g of titanium ions, the magnesium compounds containing a total of 0.1 g of magnesium ions, and the manganese compounds containing a total of 0.3 g of manganese ions were uniformly mixed to prepare the metal ion complex. 2) The reducing sugar (9.3 g), rice bran oil (49.4 g), and brown sugar (9.3 g) were uniformly mixed to prepare the composite reducing sugar preparation. 3) The composite probiotics and composite reducing sugar preparation were uniformly mixed, added to 20% water of the total mass of the composite probiotics and composite reducing sugar preparation, fermented at 37-45° C. for 3 days, and then fermented at 70-75° C. for 3-5 days to produce a fermented mixture. 4) The metal ion complex and the fermented mixture were uniformly mixed to obtain anti-viral composite formulation D, which was stored at room temperature for future use.

### Application Example 1. Influence of Different Compositions on Viral Infections within Infected Animals

### 1.1. Materials.

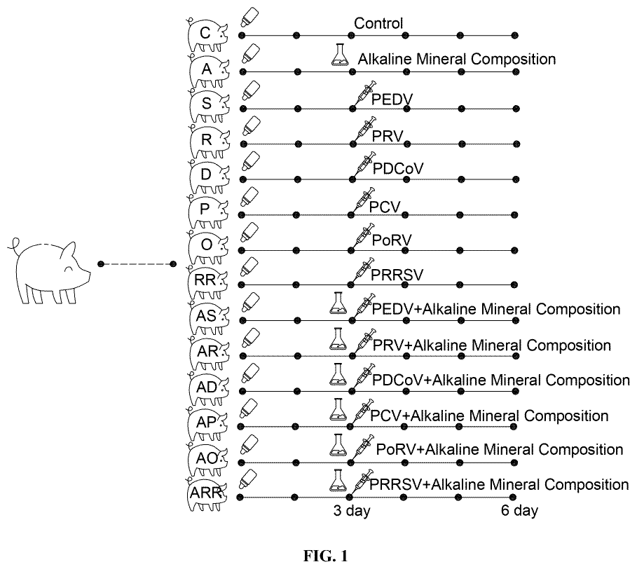

The alkaline mineral composition prepared in Example 1, purified water, virus titers as measured by 50% tissue culture infectious dose (TCID₅₀) ranging from 10^{4.2} to 10^{4.6} per 0.1 mL of PDCOV fluid, PCV fluid, PORV fluid, PRRSV fluid, and PRV fluid were utilized for the animal experiment. The animal experiment was conducted at the Experimental Animal Center of Northeast Agricultural University in Heilongjiang Province from May 1, 2023, to May 15, 2023. The experimental period lasted for 6 days, and newborn piglets were transported to the Experimental Animal Center of Northeast Agricultural University 48 hours after birth from a pig farm in Heilongjiang Province for acclimatization feeding. On the 3rd day, the piglets were grouped, and on the 6th day, samples were collected for subsequent testing at the laboratory of Professor Jinlong Li at Northeast Agricultural University. Samples of rectal swabs, serum, intestinal tissue slices, and tissue samples were collected for further analysis. The specific procedures are detailed in FIG. 1.

### 1.2. Grouping

The experimental animals were 350 newborn piglets weighing 1+0.1 kg at 1 day of age, divided into 14 groups as follows:
1) Control group, denoted as group C, where animals were fed normal water without any other treatment;
2) Administration group, denoted as group A, where the alkaline mineral composition prepared in Example 1 was diluted 1000 times with water to obtain an alkaline mineral composition water dilution solution (with a concentration of the alkaline mineral composition at 100 mg/L), which was provided as drinking water for the newborn piglets at a dosage of 100 mL/kg/d, without any other treatment;
3) to 8) Infection groups 1 to 6, denoted as groups S, R, D, P, O, and RR, respectively, where animals were orally administered 1 mL of porcine epidemic diarrhea virus (PEDV) fluid, PRV fluid, PDCOV fluid, PCV fluid, PORV fluid, PRRSV fluid, without any other treatment;
9) to 14) Administration groups 1 to 6, denoted as groups AS, AR, AD, AP, AO, and ARR, where the alkaline mineral composition prepared in Example 1 was diluted 1000 times with water to obtain an alkaline mineral composition water dilution solution (with a concentration of the alkaline mineral composition at 100 mg/L), which was provided as drinking water for the newborn piglets at a dosage of 100 mL/kg/d. Simultaneously, each piglet was orally administered 1 mL of porcine epidemic diarrhea fluid, PRV fluid, PDCOV fluid, PCV fluid, PoRV fluid, PRRSV fluid; and the experiment was quintuplicated for each group, with 5 piglets in each repeat.

### 1.3 Detection of Viral Load in Rectal Swab Samples

The rectal swabs were immersed in virus preservation solution, and the extracted viral nucleic acid was subjected to real time quantitative polymerase chain reaction (RT-qPCR) to evaluate the impact of the alkaline mineral composition on viral replication.
1) RT-qPCR assay: the rectal swab samples were subjected to nucleic acid extraction using the DNA/RNA co-extraction kit (TIANGEN, DP422, China), and RNA reverse transcription was performed using the PrimeScript™ RT reagent Kit (Perfect Real Time, RR037A, TaKaRa, Japan). Specific primers (refer to Table 1) and fluorescent dye (LightCycler®480 SYBR Green I Master, 04707516001, Roche, Switzerland) were used for quantitative polymerase chain reaction (qPCR) assay on the QuantStudio™ 5 Real-Time PCR Detection System (Thermo, USA). The qPCR system and program were conducted as per the dye instructions.

TABLE 1
Specific primers for RT-qPCR assay of the viruses

| | | | Product | |
|---|---|---|---|---|
| Virus | Primer | Sequence (5′-3′) | size | Template |
| Porcine epidemic | PEDV-F | gcacttattggcaggctttgt (SEQ | 100 bp | ORF3 |
| diarrhea | | ID NO. 1) | | |
| virus (PEDV) | PEDV-R | ccattgagaaaagaaagtgtcgtag | | |
| | | (SEQ ID NO. 2) | | |
| Porcine | PDCoV-F | agctcccaagcggactttacccaa | 112 bp | N |
| deltacoronavirus | | (SEQ ID NO. 3) | | |
| (PDCoV) | PDCoV-R | agccatacccgtcttctcagtgtc | | |
| | | (SEQ ID NO. 4) | | |
| Pseudorabies virus | PRV-F | ggtggaccggctgctgaacga | 156 bp | gD |
| (PRV) | | (SEQ ID NO. 5) | | |
| | PRV-R | gctgctggtagaacggcgtca (SEQ | | |
| | | ID NO. 6) | | |
| Porcine circovirus | PCV-F | ttgtaatgaaggtcccagcc (SEQ | 98 bp | Cap |
| (PCV) | | ID NO. 7) | | |
| | PCV-R | gaagtaacgggagtggtagg (SEQ | | |
| | | ID NO. 8) | | |
| Porcine rotavirus | PoRV-F | aactgtaaaaagctaggacc (SEQ | 106 bp | VP7 |
| (PoRV) | | ID NO. 9) | | |
| | PoRV-R | tttcggtctgcggcactg (SEQ ID | | |
| | | NO. 10) | | |
| Porcine reproductive | PRRSV-F | Ctccactacggtcaacgg (SEQ | 93 bp | M |
| and respiratory | | ID NO. 11) | | |
| syndrome virus | PRRSV-R | acaaggtttaccactccc (SEQ ID | | |
| (PRRSV) | | NO. 12) | | |

The viral load was determined by establishing standard curves using cloned plasmids containing the corresponding virus genes (refer to Table 1) purchased from SEVEN Biotech (China). The cloned plasmids were serially diluted from 101 to 108, and the results (cycle threshold (CT) values) obtained after each dilution were detected by RT-qPCR. The dilution factor was plotted on the x-axis, and the CT value was plotted on the y-axis to establish standard curves for different viruses. The standard curves for PEDV, PDCOV, PCV, PORV, PRRSV, and PRV were established as follows: y=3.647x+6.146, y=4.564x−5.260, y=3.711x+1.953, y=4.234x−3.461, y=4.147x+3.156, and y=4.906x−1.230, respectively. The viral load of the test samples was determined based on the CT values obtained from RT-qPCR according to the standard curves.

The results of the viral load detection are shown in FIG. 2 and Table 2. Compared to that in group S, the porcine epidemic diarrhea viral load in group AS was significantly reduced (p≤0.0001). Compared to that in group R, the pseudorabies viral load in group AR was significantly reduced (p≤0.0001). Compared to that in group D, the porcine deltacoronaviral load in group AD was significantly reduced (p≤0.001). Compared to that in group P, the porcine circoviral load in group AP was significantly reduced (p≤0.001). Compared to that in group O, the porcine rotaviral load in group AO was significantly reduced (p≤0.0001). Compared to that in group RR, the porcine reproductive and respiratory syndrome viral load in group ARR was significantly reduced (p≤0.0001).

TABLE 2
Results of viral load assay

| Group | | | | | | | | | | | | | | |
|---|---|---|---|---|---|---|---|---|---|---|---|---|---|---|
| (copies/μL) | C | A | S | R | D | P | O | RR | AS | AR | AD | AP | AO | ARR |
| PEDV | / | / | 1.183 × | / | / | / | / | / | 2.979 × | / | / | / | / | / |
| | | | 10⁶ | | | | | | 10⁵ | | | | | |
| PRV | / | / | / | 3.385 × | / | / | / | / | / | 1.594 × | / | / | / | / |
| | | | | 10⁵ | | | | | | 10⁵ | | | | |
| PDCoV | / | / | / | / | 6.386 × | / | / | / | / | / | 1.240 × | / | / | / |
| | | | | | 10⁵ | | | | | | 10⁵ | | | |
| PCV | / | / | / | / | / | 1.318 × | / | / | / | / | / | 9.214 × | / | / |
| | | | | | | 10⁵ | | | | | | 10⁴ | | |
| PoRV | / | / | / | / | / | / | 3.373 × | / | / | / | / | / | 1.132 × | / |
| | | | | | | | 10⁶ | | | | | | 10⁶ | |
| PRRSV | / | / | / | / | / | / | / | 1.225 × | / | / | / | / | / | 5.883 × |
| | | | | | | | | 10⁶ | | | | | | 10⁵ |

The results indicate that the alkaline mineral composition as described in the present disclosure significantly inhibits the infection of PEDV, PRV, PDCOV, PCV, PORV, and PRRSV in the animal body.

### Application Example 2. Effects of Different Compositions on Gene Expression and Metabolic Regulation in Infected Animal Organisms

The collected colon samples from Application Example 1 were washed with normal saline to remove bloodstains and impurities, and non-essential tissues were removed. The samples were dried, cut into small pieces, and placed in pre-labeled 1.5 mL EP tubes. The tubes were snap-frozen in liquid nitrogen for subsequent experiments.

### 2.1 Transcriptomics and Metabolomics Assay and Analysis:

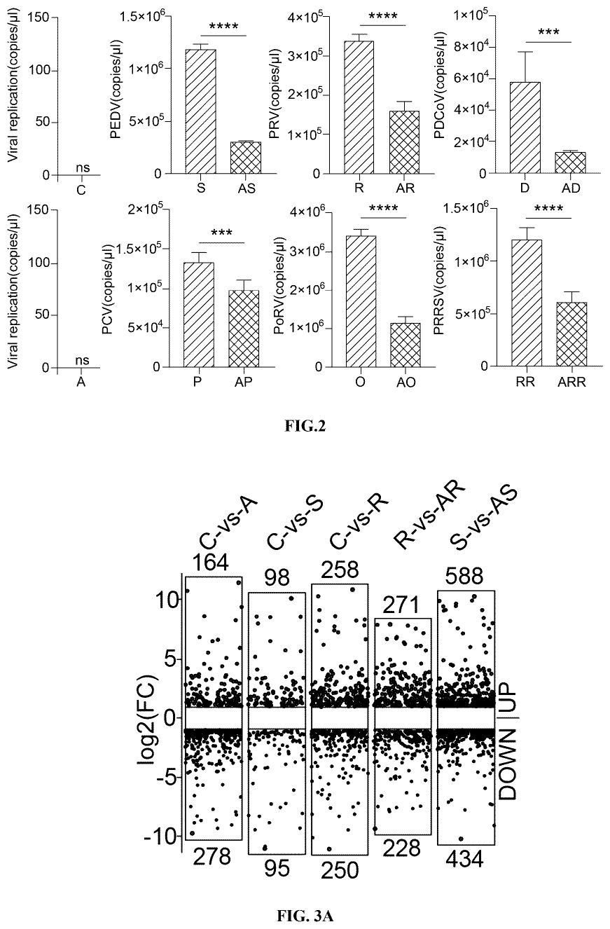

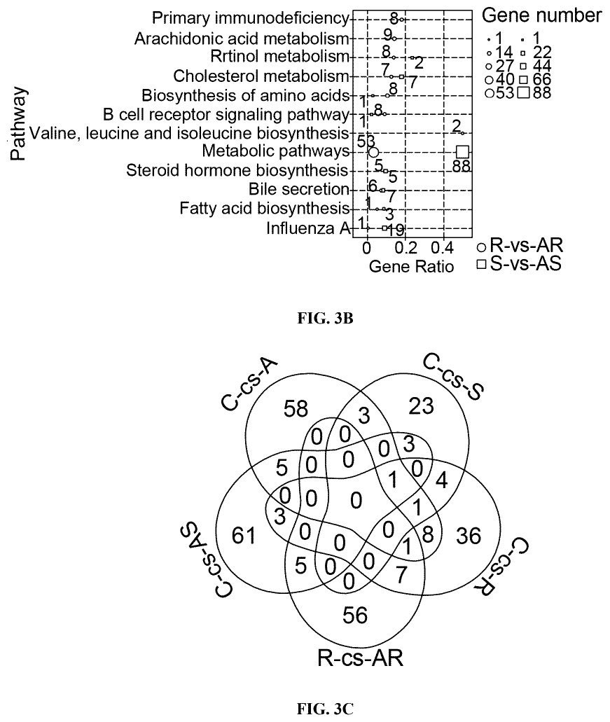

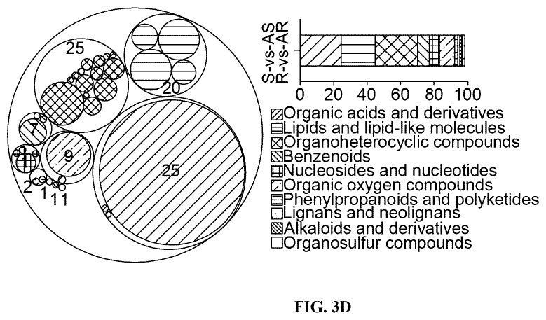

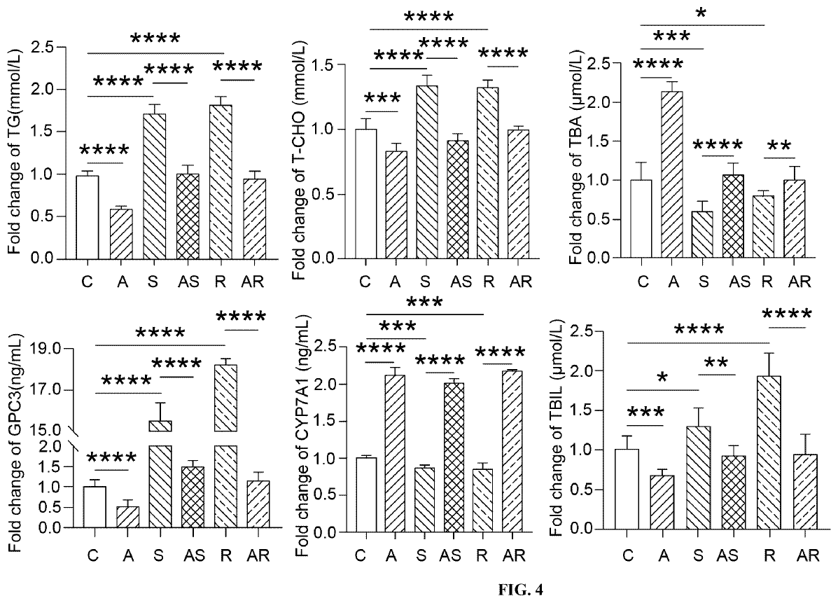

The analysis was conducted by Genedenovo Biotechnology Co., Ltd in Guangzhou, China, to evaluate the effects of the composition on gene expression and metabolic regulation in newborn piglets. The impact on gene expression levels in piglets of different groups (group C, group A, group S, group R, group AS, and group AR) is shown in FIG. 3A: Compared to group C, the A group showed a total of 442 differentially expressed genes (DEGs), with 164 upregulated and 278 downregulated. Compared to group C, group S showed 193 DEGs, with 98 upregulated and 95 downregulated. Compared to group C, group R showed 508 DEGs, with 258 upregulated and 250 downregulated. Compared to group S, group AS showed 1022 DEGs, with 588 upregulated and 434 downregulated. Compared to group R, group AR showed 499 DEGs, with 271 upregulated and 228 downregulated. The enrichment results of the DEGs between group S and group AS, and those between group R and group AR are shown in FIG. 3B, where the DEGs were mainly enriched in metabolic pathways, bile secretion, and cholesterol metabolism. The effects on metabolic expression levels in piglets of different groups (group C, A group, group S, group R, group AS, and group AR) are shown in FIG. 3C. Compared to group C, group A showed a total of 76 differentially expressed metabolites (DEMs). Compared to group C, group S showed 35 DEMs. Compared to group C, group R showed 55 DEMs. Compared to group S, group AS showed 78 DEMs. Compared to group R, group AR showed 69 DEMs. The classification results of the DEMs between group S and group AS, and those between group R and group AR are shown in FIG. 3D, where the DEMs were mainly enriched in organic acids and derivatives, lipids and lipid-like molecules, and organoheterocyclic compounds. From the above results, it can be seen that the alkaline mineral composition as described in the present disclosure significantly affects lipid metabolism and ion homeostasis in piglets.

### 2.2 Detection of Serum Biochemical Index.

The serum collected in Application Example 1 was used as the test sample for biochemical index detection. The reagents for testing cholesterol 7-a-hydroxylase (H461-1), total bilirubin (C019-1-1), total bile acids (E003-2-1), alkaline phosphatase (A059-2-2), glypican-3 (H455-1), triglycerides (A110-1-1), and total cholesterol (A111-1-1) were purchased from Nanjing Jiancheng Bioengineering Institute, Jiangsu, China. The corresponding detection methods were carried out according to the manufacturer's instructions to evaluate the impact of the compositions on lipid-related metabolites in newborn piglets. The results of the serum lipid-related biochemical index detection are shown in FIG. 4 and Table 3, indicating that compared to those of group C, the triglycerides, glypican-3, total cholesterol, and total bile acids in groups S and R were significantly elevated (p≤0.05 or p≤0.0001), while cholesterol 7α-hydroxylase and total bilirubin were significantly decreased (p≤0.05 or p≤0.001). Compared to those of groups S and R, the triglycerides, glypican-3, total cholesterol, and total bile acids in groups AS and AR were significantly decreased (p≤0.01 or p≤0.0001), while cholesterol 7α-hydroxylase and total bilirubin were significantly increased (p≤0.01 or p≤0.0001). These results indicate that the alkaline mineral composition in the present disclosure significantly inhibits virus-mediated host lipid metabolism regulation.

TABLE 3
Results of serum lipid-related biochemical index detection

| Index | Group C | Group A | Group S | Group AS | Group R | Group AR |
|---|---|---|---|---|---|---|
| TG (mmol/L) | 1.482 | 0.921 | 2.584 | 1.523 | 2.739 | 1.418 |
| T-CHO (mmol/L) | 4.095 | 3.392 | 5.470 | 3.738 | 5.416 | 4.070 |
| TBA (μmol/L) | 0.392 | 0.836 | 0.235 | 0.419 | 0.309 | 0.393 |
| GPC3 (ng/mL) | 0.567 | 0.296 | 8.766 | 0.841 | 10.327 | 0.651 |
| CYP7A1 (ng/mL) | 1.277 | 0.622 | 0.977 | 2.567 | 0.971 | 2.774 |
| TBIL (μmol/L) | 25.839 | 16.944 | 31.911 | 21.918 | 49.711 | 23.276 |

T 2.3. Iodine-Schiff staining and Oil Red staining:

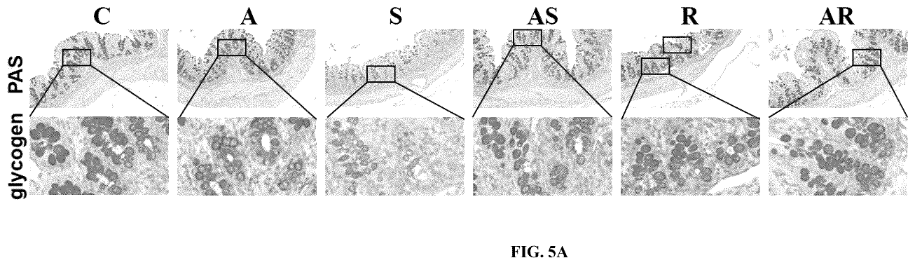

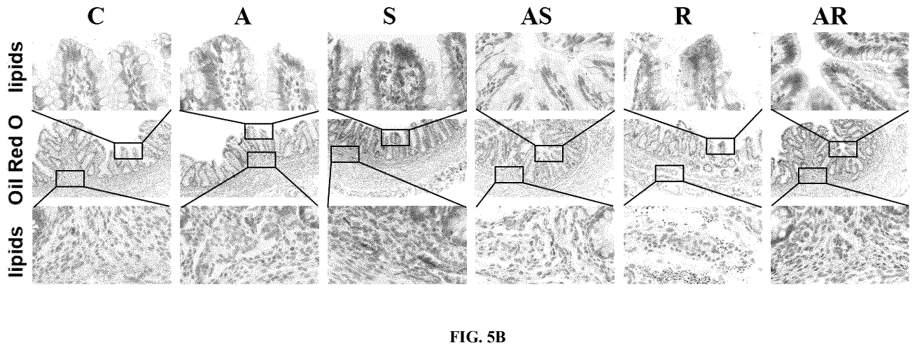

The colon samples collected in Application Example 1 were fixed in a 4% polyoxymethylene (P0099, Beyotime, China) and glutaraldehyde (354400, Merck, USA) solution. Subsequently, iodine-Schiff staining and oil red staining were performed by Hangzhou Powerful Biology Co., Ltd. to evaluate changes in intestinal glycolipid metabolism. The results of the iodine-Schiff staining are shown in FIG. 5A. The results indicate that compared to group C, there was a reduction in glycogen in group S; compared to group S, there was an increase in glycogen in group AS; compared to group C, there was no significant change in glycogen in group R and group AR. The results of the oil red staining are shown in FIG. 5B. Compared to group C, there was a decrease in lipids in the administration groups; compared to group C, there was an increase in lipids in group S and group R; compared to group S and group R, there was a decrease in lipids in group AS and group AR. It can be observed that the alkaline mineral composition in the present disclosure regulates virus-mediated lipid metabolism universally and selectively affects virus-mediated glucose metabolism.

### Application Example 3. Effects of Different Compositions on the Morphology of Infected Animal Intestinal Tissues

### 3.1 Hematoxylin-Eosin (H&E) Staining:

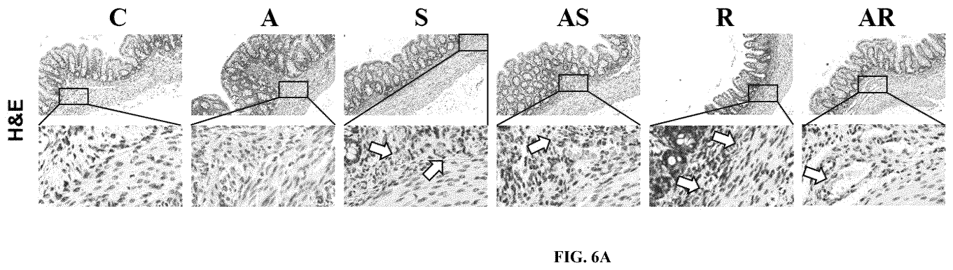

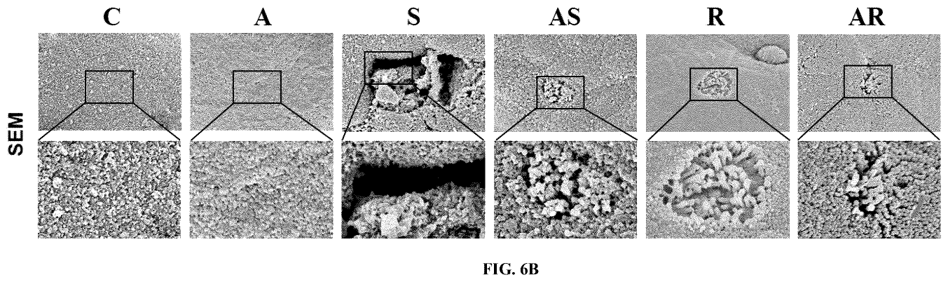

The colon samples collected in Application Example 1 were fixed in a 4% polyoxymethylene and glutaraldehyde solution for H&E staining. The height of the villi, depth of the crypts, thickness of the mucosa, muscle layer, serosa, and the density of intestinal villi observed under a scanning electron microscope were evaluated to assess the protective effect of the composition on the physical barrier of the intestines in newborn piglets. H&E staining was performed by Hangzhou Powerful Biology Co., Ltd. The results of the H&E staining, as shown in FIG. 6A, indicate that group C, group A, group S, group R, group AS, and group AR had different effects on the morphological structure of the intestinal tissues in newborn piglets. Compared to group C, group A showed better intestinal villi morphology with tighter microvilli. Compared to group S and group R, group AS and group AR had less severe damage to the intestinal villi morphology and U-shaped crypts.

### 3.2 Scanning Electron Microscopy (SEM) Detection:

The colon samples collected in Application Example 1 were fixed in glutaraldehyde at 4° C. for 1.5 hours. The samples were then washed with phosphate-buffered saline (PBS), dehydrated using a series of ethanol solutions (50%, 70%, 90%, and 100%), followed by dehydration with tert-butanol. The samples were dried overnight at −20° C., mounted on stubs, coated, and observed under an electron microscope (HITACHI S-3400N). The results of the SEM, as shown in FIG. 6B, indicate that compared to group C, group S, and group R showed defects in the intestinal villi and damage to the intestinal barrier. Compared to group S and group R, group AS and group AR showed reduced damage to the intestinal villi. It can be observed that the alkaline mineral composition in the present disclosure has a good protective effect on the intestinal barrier, helping the host resist the damage caused by viruses to the body.

### Application Example 4: Effects of Different Compositions on Viral Replication and Infection in Cells Infected with Different Viruses

### 4.1 Materials

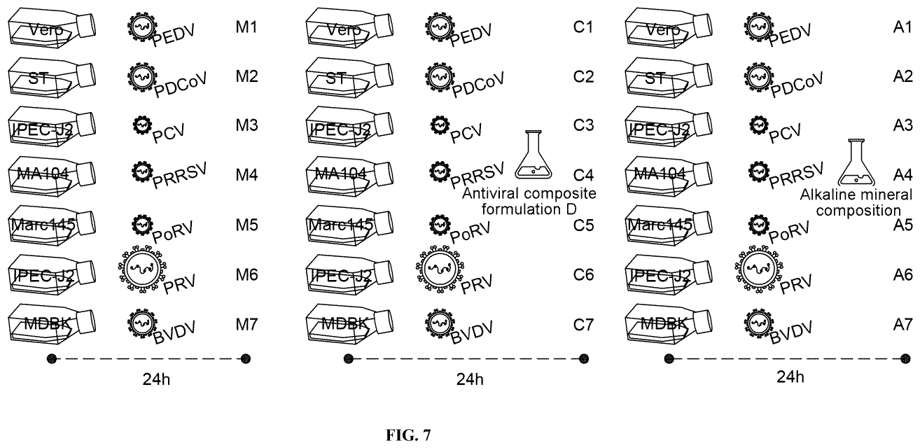

The alkaline mineral composition prepared in Example 1, water, virus fluid (PEDV fluid, PDCOV fluid, PCV fluid, PORV fluid, PRRSV fluid, PRV fluid, and bovine viral diarrhea virus (BVDV) fluid, with virus titers as measured by TCID₅₀ ranging from 10^{4.2} to 10^{4.6} per 0.1 mL), and the antiviral composite formulation D prepared in Comparative Example 5 were used for cell experiments. The cell experiments were conducted at the Key Laboratory of Pathogenic Mechanisms and Comparative Medicine of Animal Diseases in Heilongjiang Province from Sep. 1, 2022, to Jun. 1, 2023. The cell lines used were as follows: Vero cells for PEDV, ST cells for PDCOV and PRV, IPEC-J2 cells for PCV, MA104 cells for PORV, Marc145 cells for PRRSV, and MDBK cells for BVDV. The cell concentrations ranged from 1.8×10⁶ to 2×10⁶ cells/mL. The cell experiments lasted for 48 hours, with the cells thawed from liquid nitrogen, passaged, and cultured in T25 cell culture vessels until reaching 80% confluence. The alkaline mineral composition prepared in Example 1 at a dilution factor of 1.4×10⁵ or an equal mass of the antiviral composite formulation D prepared in Comparative Example 5 was added to the cells and incubated for 24 hours. Subsequently, the respective susceptible cells were infected with the above-mentioned viruses, and the supernatant and cell samples were collected 24 hours post-infection for further analysis. The specific process is detailed in FIG. 7.

### 4.2 Grouping

The above cells in 189 T25 cell culture vessels (3289, Corning, USA) were subjected to a total of 21 groups of treatments:

Infection Group 1 (MI): African green monkey kidney (Verda reno, Vero) cells, treated with water and infected with 0.1 mL of PEDV without any additional treatment.

Infection Group 2 (M2): swine testis (ST) cells, treated with water and infected with 0.1 mL of PDCoV without any additional treatment.

Infection Group 3 (M3): cells from intestinal porcine epithelial cell (IPEC)-J2, treated with water and infected with 0.1 mL of PCV without any additional treatment.

Infection Group 4 (M4): African green monkey embryonic kidney (microbiological associates-104, MA104) cells, treated with water and infected with 0.1 mL of PoRV without any additional treatment.

Infection Group 5 (M5): African green monkey embryonic kidney cells (meat animal research center-145, Marc145), treated with water and infected with 0.1 mL of PRRSV without any additional treatment.

Infection Group 6 (M6): IPEC-J2 cells, treated with water and infected with 0.1 mL of PRV without any additional treatment.

Infection Group 7 (M7): Madin-Darby bovine kidney (MDBK) cells, treated with water and infected with 0.1 mL of bovine viral diarrhea virus (BVDV) without any additional treatment.

Control Group 1 (C1): Vero cells, treated with a diluted solution of antiviral composition D (prepared in Example 5) at a 1.4×10⁵ dilution, and infected with 0.1 mL of PEDV without any additional treatment.

Control Group 2 (C2): ST cells, infected with 0.1 mL of PDCOV, with the same treatment as Group C1.

Control Group 3 (C3): IPEC-J2 cells, infected with 0.1 mL of PCV, with the same treatment as Group C1.

Control Group 4 (C4): MA104 cells, infected with 0.1 mL of PORV, with the same treatment as Group C1.

Control Group 5 (C5): Marc145 cells, infected with 0.1 mL of PRRSV, with the same treatment as Group C1.

Control Group 6 (C6): IPEC-J2 cells, infected with 0.1 mL of PRV, with the same treatment as Group C1.

Control Group 7 (C7): MDBK cells, infected with 0.1 mL of BVDV, with the same treatment as Group C1.

Experimental Group 1 (A1): Vero cells, treated with a diluted solution of the alkaline mineral composition prepared in Example 1 at a 1.4×10⁵ dilution, and infected with 0.1 mL of PEDV.

Experimental Group 2 (A2): ST cells, infected with 0.1 mL of PDCOV, with the same treatment as Group A1.

Experimental Group 3 (A3): IPEC-J2, infected with 0.1 mL of PCV, with the same treatment as Group A1.

Experimental Group 4 (A4): MA104 cells, infected with 0.1 mL of PORV, with the same treatment as Group A1.

Experimental Group 5 (A5): Marc145 cells, infected with 0.1 mL of PRRSV, with the same treatment as Group A1.

Experimental Group 6 (A6): IPEC-J2 cells, treated with a diluted solution of the alkaline mineral composition prepared in Example 1 at a 1.4×10⁵ dilution, and infected with 0.1 mL of PRV.

Experimental Group 7 (A7): MDBK cells, treated with a diluted solution of the alkaline mineral composition prepared in Example 1 at a 1.4×10⁵ dilution, and infected with 0.1 mL of bovine viral diarrhea virus (BVDV).

The experiment was triplicated for each group, with three bottles of cells for each replicate.

### 4.3 RT-qPCR Assay

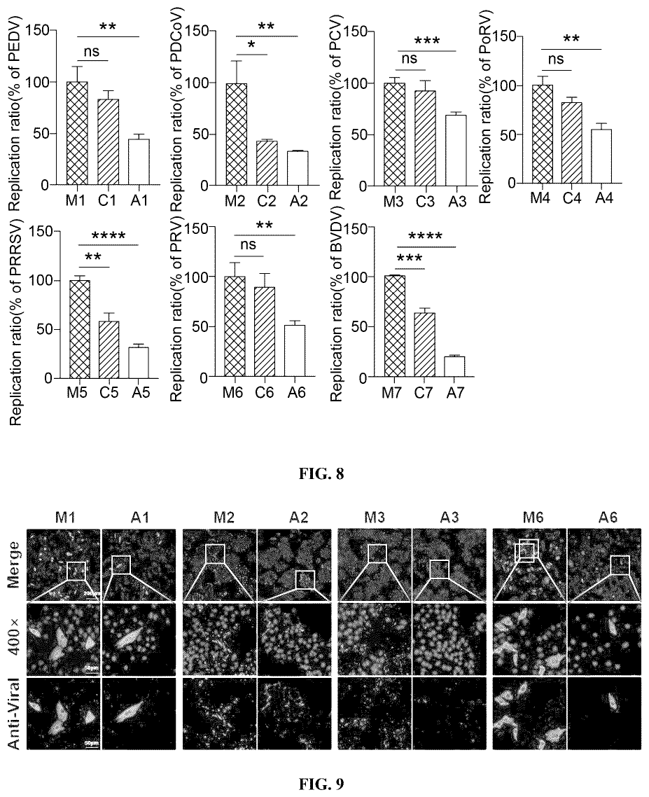

0.5 mL of cell culture supernatant samples from each group were aspirated using a pipette and placed into pre-labeled 1.5 mL EP tubes. Nucleic acid extraction was performed for virus gene transcription level detection to assess the impact of the compositions on viral replication ratio, using the same reagent and method as described in Application Example 1. The viral replication levels in the various infection groups, control groups, and experimental groups are shown in FIG. 8 and Table 4. Compared to that in group M1, the replication ratio of PEDV in group A1 significantly decreased (p≤0.01), while there was no significant change in group C1 (p>0.05); compared to that in group M2, the replication ratio of PDCOV in group A2 significantly decreased (p≤0.01), while it decreased in group C2 (p≤0.05); compared to that in group M3, the replication ratio of PCV in group A3 significantly decreased (p≤0.001), while there was no significant change in group C3 (p>0.05); compared to that in group M4, the replication ratio of PoRV in group A4 significantly decreased (p≤0.05), while there was no significant change in group C4 (p>0.05); compared to that in group M5, the replication ratio of PRRSV in group A5 significantly decreased (p≤0.0001), while it decreased significantly in group C5 (p≤0.05); compared to that in group M6, the replication ratio of PRV in group A6 significantly decreased (p≤0.01), while there was no significant change in group C6 (p>0.05); compared to that in group M7, the replication ratio of BVDV in group A7 significantly decreased (p≤0.0001), while it decreased significantly in group C7 (p≤0.001).

TABLE 4
Viral replication

| Viral | | | | | | | |
|---|---|---|---|---|---|---|---|
| replication | | | | | | | |
| (copies/μL) | PEDV | PDCoV | PCV | PoRV | PRRSV | PRV | BVDV |
| M1 | 6.275 × 10⁴ | / | / | / | / | / | / |
| M2 | / | 5.360 × 10⁴ | / | / | / | / | / |
| M3 | / | / | 6.844 × 10⁴ | / | / | / | / |
| M4 | / | / | / | 8.827 × 10⁴ | / | / | / |
| M5 | / | / | / | / | 1.450 × 10⁵ | / | / |
| M6 | / | / | / | / | / | 4.044 × 10⁴ | / |
| M7 | | | | | | | 2.892 × 10⁴ |
| C1 | 5.415 × 10⁴ | / | / | / | / | / | / |
| C2 | / | 2.331 × 10⁴ | / | / | / | / | / |
| C3 | / | / | 6.328 × 10⁴ | / | / | / | / |
| C4 | / | / | / | 7.350 × 10⁴ | / | / | / |
| C5 | / | / | / | / | 7.719 × 10⁴ | / | / |
| C6 | / | / | / | / | / | 3.628 × 10⁴ | / |
| C7 | | | | | | | 1.808 × 10⁴ |
| A1 | 2.542 × 10⁴ | / | / | / | / | / | / |
| A2 | / | 1.772 × 10⁴ | / | / | / | / | / |
| A3 | / | / | 4.711 × 10⁴ | / | / | / | / |
| A4 | / | / | / | 4.555 × 10⁴ | / | / | / |
| A5 | / | / | / | / | 4.351 × 10⁴ | / | / |
| A6 | / | / | / | / | / | 2.070 × 10⁴ | / |
| A7 | | | | | | | 5.623 × 10³ |

As can be seen, the alkaline mineral composition of the present disclosure significantly inhibited the efficiency of viral replication and was superior to the antiviral combination preparation D prepared in comparative Example 5.

### 4.4 Immunofluorescence Assay

After washed with phosphate buffer, the cell slides were fixed in 4% paraformaldehyde solution for 10 minutes, permeabilized with TritonX-100 (A16046, Thermo Fisher Scientific, USA) for 10 minutes, blocked with 3% bovine serum albumin (BSA, BS114, Biosharp, China) for 1 hour, incubated with antibodies against PEDV (SD17-103, Medgene Labs, USA) or PDCOV (SD55-197, Medgene Labs, USA) or PCV (self-made by the Key Technology Innovation Team for the Prevention and Control of Group Diseases in Livestock and Poultry at Northeast Agricultural University) or PRV (gifted by the Harbin Veterinary Research Institute, Chinese Academy of Agricultural Sciences) for 1 hour, and incubated with Alexa Fluor 488-labeled goat anti-mouse IgG (A0428, Beyotime, China) for 30 minutes in the dark. Observation was carried out using a fluorescence microscope (THUNDER, Leica, Germany) to evaluate the effect of the combination on intracellular virus infection. The intracellular virus infection of each infection group and experimental group is shown in FIG. 9, where compared to group M1, group A1 showed a decrease in PEDV infection; compared to group M2, group A2 showed a decrease in PDCoV infection; compared to group M3, group A3 showed a decrease in PCV infection; and compared to group M6, group A6 showed a decrease in PRV infection. This indicates that the alkaline mineral composition of the present disclosure can inhibit virus infection within cells.

### Application Example 5: Effects of Different Compositions on Lipid Content in Animal Cells Infected with Different Viruses

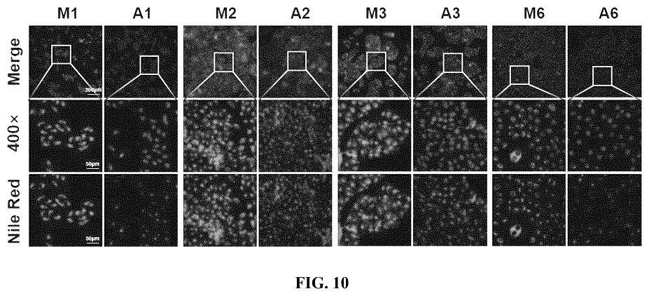

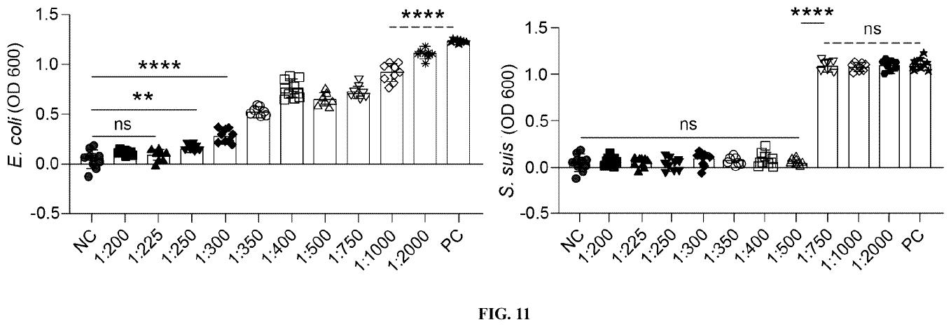

Lipid droplet red fluorescence detection was used to evaluate the lipid content in animal cells infected with different viruses. Cell samples were obtained from cell slides in T25 cell culture vessels (BS-14-RC, Biosharp, China), washed with phosphate-buffered saline (PBS), and stained with the lipid droplets red fluorescence assay kit with nile red (C2051M, Beyotime, China) for evaluation of the impact of the compositions on intracellular lipids. The lipid content in cells from each infection group and experimental group is shown in FIG. 10, where compared to group MI, group A1 showed a decrease in lipid content; compared to group M2, group A2 showed a decrease in lipid content; compared to group M3, group A3 showed a decrease in lipid content; and compared to group M6, group A6 showed a decrease in lipid content. It can be observed that the alkaline mineral composition of the present disclosure significantly inhibits the virus-induced host lipid metabolism, leading to a reduction in lipid content.

### Application Example 6: Effects of Different Compositions on Bacterial Reproduction Efficiency

### 6.1 Materials

The alkaline mineral composition prepared in Example 1 was used for antibacterial testing at the Key Laboratory of Pathogenic Mechanisms and Comparative Medicine of Animal Diseases in Heilongjiang Province, from Jul. 1, 2022, to Sep. 1, 2022. The bacteria used in the experiment included Streptococcus suis (S. suis) and Escherichia coli (E. coli), with a bacterial density of 1.0 as measured by optical density (OD). Each bacterial test involved 108 bacteria in a 10 mL shaking tube containing LB medium (10855001, Thermo, USA).

### 6.2 Grouping

6.2.1 Grouping in S. suis Test

A total of 12 treatment groups were established:
1) negative control group, denoted as NC, treated with LB medium without any other treatment;
2) treatment group 1, denoted as 1:200, treated with LB medium containing the alkaline mineral composition prepared in Example 1 diluted to 5 mg in 200 times the volume of water (1 mL), with a dilution ratio of 1:200, and infected with 0.1 mL of S. suis, without any other treatment;
3) to 11) treatment groups 2 to 10, denoted as 1:225, 1:250, 1:300, 1:400, 1:500, 1:750, 1:1000, 1:2000, with the only difference from treatment group 1 being the dilution ratio of the combination, which was 225, 250, 300, 350, 400, 500, 750, 1000, and 2000 times, respectively;
12) positive control group, denoted as PC, treated with LB medium (10855001, Thermo, USA) containing a strain of S. suis with a bacterial density of 1.0 as measured in OD; and the test was triplicated for each group, with 3 tubes per replicate.
   6.2.2 Grouping in E. coli Test

A total of 12 treatment groups were established:

Groups 13) to 24). The treatments corresponded to groups 1) to 12) of the S. suis test, with the only difference being the replacement of S. suis with E. coli.

### 6.3 Bacterial Density Detection

The experiment lasted for 12 hours. After the experimental bacteria were resuscitated and stabilized from the ultra-low temperature freezer, bacterial liquid was obtained. Five milligrams of the alkaline mineral composition prepared in Example 1 was added to 10 mL of LB medium according to the corresponding dilution ratio, followed by addition of 0.1 mL of S. suis or E. coli. After 12 hours, bacterial liquid samples were collected for subsequent detection. Two microlitters of bacterial liquid samples from each group were aspirated with a pipette and placed in a calibrated ultravilet (UV) spectrophotometer for bacterial density detection, in order to evaluate the impact of the inhibitor on bacterial reproduction.

The bacterial reproduction of each control group and treatment group is shown in FIG. 11 and Table 5, respectively. Compared to the positive control group, the density of S. suis in the 1:200 to 1:500 groups decreased significantly (p≤0.0001); and compared to the positive control group, the density of E. coli in the 1:200 to 1:1000 groups decreased significantly (p≤0.0001). It can be observed that the alkaline mineral composition of the present disclosure significantly inhibits bacterial reproduction.

TABLE 5
Bacterial reproduction.

| Group | NC | 1:200 | 1:225 | 1:250 | 1:300 | 1:350 | 1:400 | 1:500 | 1:750 | 1:1000 | 1:2000 | PC |
|---|---|---|---|---|---|---|---|---|---|---|---|---|
| E. Coli | 0.045 | 0.110 | 0.088 | 0.159 | 0.281 | 0.524 | 0.758 | 0.646 | 0.715 | 0.915 | 1.107 | 1.234 |
| (OD) | | | | | | | | | | | | |
| S. Suis | 0.030 | 0.064 | 0.054 | 0.024 | 0.082 | 0.067 | 0.082 | 0.063 | 1.083 | 1.079 | 1.096 | 1.106 |
| (OD) | | | | | | | | | | | | |

### Application Example 7: Verification Test for Synergistic Effect

### 7.1 Materials

Water-diluted fluids of the alkaline mineral composition A prepared in Example 1, the alkaline mineral composition B prepared in Example 2, the alkaline mineral composition C prepared in Example 3, the single-component material A1 consisting of the metal ion complex prepared in Comparative Example 1, the single-component material A2 consisting of the alkaline mineral complex prepared in Comparative Example 2, the single-component material A3 consisting of the alkaline soluble rare earth salt prepared in Comparative Example 3, and the single-component material A4 consisting of the composite gel prepared in Comparative Example 4 (0.25 L for each, with a mass ratio of 1:1.4×10⁵ of each material to water) as well as the virus fluids (PEDV, PDCOV, PCV, PORV, porcine respiratory and reproductive syndrome virus, and PRV, with virus titers ranging from 10^{4.2} to 10^{4.6} per 0.1 mL as measured by TCID₅₀) were used for component testing at the Key Laboratory of Pathogenic Mechanisms and Comparative Medicine of Animal Diseases in Heilongjiang Province, from Apr. 1, 2022, to May 1, 2022. The experimental cells were Vero cells with a concentration of 2×10⁶ cells/mL.

### 7.2 Grouping

A total of 54 T25 cell culture vessels were divided into 6 treatment groups:
1) positive control group, denoted as PC, Vero cells infected with 0.1 mL of PEDV without any other treatment;
2) group single-component material A1 consisting of the metal ion composite, denoted as A1, treated with the water dilution of the single-component material A1 of the metal ion composite, with other treatments same as the PC group;
3) group single-component material A2 consisting of the alkaline mineral composite, denoted as A2, treated with the water dilution of the single-component material A2 of the alkaline mineral composite, with other treatments same as the A1 group;
4) group single-component material A3 consisting of the alkaline soluble rare earth salt, denoted as A3, treated with the water dilution of the single-component material A3 of the alkaline soluble rare earth salt, with other treatments same as the A1 group;
5) group single-component material A4 consisting of the composite gel, denoted as A4, treated with the water dilution of the single-component material A4 of the composite gel, with other treatments same as the A1 group;
6) group alkaline mineral composition A, denoted as A, treated with the water dilution of the alkaline mineral composition prepared in Example 1, with other treatments same as the A1 group;
7) group alkaline mineral composition B, denoted as B, treated with the water dilution of the alkaline mineral composition prepared in Example 2, with other treatments same as the A1 group;
8) group alkaline mineral composition C, denoted as C, treated with the water dilution of the alkaline mineral composition prepared in Example 3, with other treatments same as the A1 group.

The test was triplicated for each group, with 3 vessels per replicate.

### 7.3 Viral Gene Transcription Level Detection

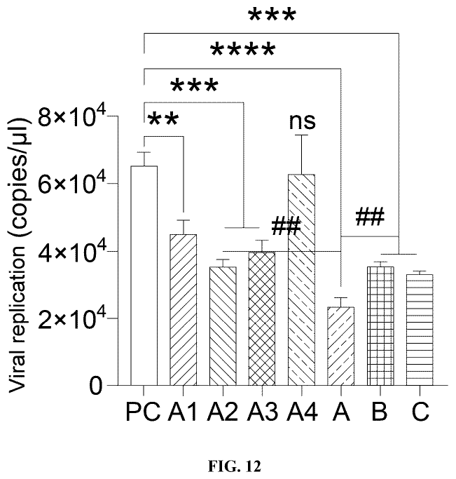

The experiment lasted for 48 hours. After the experimental cells were resuscitated from liquid nitrogen and passaged to a stable generation, they were cultured in T25 cell culture vessels until reaching a confluence of 80%. The above components were added and incubated for 24 hours, and supernatant samples were collected for subsequent testing. 0.5 mL of culture medium from each group was aspirated with a pipette and placed in pre-labeled 1.5 mL EP tubes. Nucleic acid was extracted for viral gene transcription level detection using the RT-qPCR assay method consistent with Application Example 1, to evaluate the impact of different compositions on viral replication. The viral replication of each group is shown in FIG. 12 and Table 6. Compared to that in the PC group, the content of PEDV decreased significantly (p≤0.01) in group A1, decreased extremely significantly in group A2 (p≤0.001), decreased extremely significantly in group A3 (p≤0.001), showed no significant change in group A4 (p>0.05), decreased extremely significantly in group A (p≤0.0001), and decreased extremely significantly in groups B and C (p≤0.001). Additionally, compared to the A2 group, which had the best single-component effect, groups A, B, and C showed a significant decrease in the content of PEDV (p≤0.01). Furthermore, compared to that in group B or C, the content of PEDV in group A decreased significantly (p≤0.01).

TABLE 6
Viral replication

| Group | PC | A1 | A2 | A3 | A4 | A | B | C |
|---|---|---|---|---|---|---|---|---|
| Viral | 6.275 × | 4.483 × | 3.522 × | 3.993 × | 6.524 × | 2.314 × | 3.526 × | 3.294 × |
| replication | 10⁴ | 10⁴ | 10⁴ | 10⁴ | 10⁴ | 10⁴ | 10⁴ | 10⁴ |
| (copies/μL) | | | | | | | | |

As can be seen, the alkaline mineral composition used for antiviral purposes in the present disclosure exhibits a synergistic effect among its four components, and the combined effect is greater than the individual effects of each component when used alone.

In summary, the combination of the present disclosure can regulate lipid metabolism in the body, inhibit viral replication and transmission, and enhance intestinal barrier function. The alkaline mineral composition effectively inhibits viral infections in piglets. Moreover, the metal ion complex in the alkaline mineral composition of the present disclosure can maintain internal water balance, support energy metabolism and does not produce toxic side effects or resistance. Since the digestive and immune systems of piglets are not fully developed, supplementing with mineral complexes can enhance antioxidant activity and promote immune system function. Additionally, mineral complexes can regulate metabolism and body development, improve intestinal morphology, and reduce cholesterol levels. The alkaline rare earth elements in the alkaline mineral composition promote blood circulation and act as toxin filters. Furthermore, using a composite gel as the base for the alkaline mineral composition can strengthen the intestinal barrier, inhibit harmful bacterial growth, and facilitate storage. Therefore, it may serve as a vaccine alternative for antiviral purposes.

Although the above examples provide a detailed description of the present disclosure, they represent only some embodiments and not all embodiments of the present disclosure. Other embodiments can be obtained based on the present examples without inventive effort, and all such embodiments are within the protection scope of the present disclosure.

What is claimed is:

1. An alkaline mineral composition, comprising the following components in parts by weight: 60-70 parts of metal ion complex, 25-30 parts of mineral ion complex, 1-9.5 parts of alkaline soluble rare earth salt, and 0.5-4 parts of composite gel.

2. The alkaline mineral composition of claim 1, wherein the metal ion complex comprises a sodium compound, a potassium compound, a zinc compound, and an iron compound; sodium ions, potassium ions, zinc ions, and iron ions in the sodium compound, the potassium compound, the zinc compound, and the iron compound have a mass ratio of 16.48-19.02:41.21-43.18:0.23-0.25:2.06-7.93; the sodium compound comprises sodium chloride, sodium carbonate, sodium selenate, and sodium metasilicate; the potassium compound comprises potassium chloride, potassium iodide, and potassium carbonate; the zinc compound comprises zinc gluconate; and the iron compound comprises ferrous sulfate, ferrous lactate, and ferrous gluconate.

3. The alkaline mineral composition of claim 1, wherein the mineral ion complex comprises a selenium compound, an iodine compound, and a silicon compound; selenium ions, iodine ions, and silicon ions in the selenium compound, the iodine compound, and the silicon compound have a mass ratio of 1.14-4.67:18.66-25:1.14-4.67; the selenium compound comprises sodium selenate; the iodine compound comprises potassium iodide; and the silicon compound comprises sodium metasilicate.

4. The alkaline mineral composition of claim 1, wherein the alkaline soluble rare earth salt comprises digermanate and lanthanum carbonate; the digermanate and the lanthanum carbonate have a mass ratio of 0.87-3.8:0.13-5.7.

5. The alkaline mineral composition of claim 1, wherein the composite gel comprises sodium alginate and nano silver; the sodium alginate and the nano silver have a mass ratio of 0.48-3:0.02-1.

6. A method for preparing the alkaline mineral composition of claim 1, comprising the following steps: dissolving the metal ion complex, the mineral ion complex, and the alkaline soluble rare earth salt successively in the composite gel, and performing ionization to obtain the alkaline mineral composition.

7. The method of claim 6, wherein the ionization is performed at a temperature of 25-35° C. and an ionization potential of −25 to 25 mV for 1-2 days.

8. The method of claim 6, wherein the metal ion complex comprises a sodium compound, a potassium compound, a zinc compound, and an iron compound; sodium ions, potassium ions, zinc ions, and iron ions in the sodium compound, the potassium compound, the zinc compound, and the iron compound have a mass ratio of 16.48-19.02:41.21-43.18:0.23-0.25:2.06-7.93; the sodium compound comprises sodium chloride, sodium carbonate, sodium selenate, and sodium metasilicate; the potassium compound comprises potassium chloride, potassium iodide, and potassium carbonate; the zinc compound comprises zinc gluconate; and the iron compound comprises ferrous sulfate, ferrous lactate, and ferrous gluconate.

9. The method of claim 6, wherein the mineral ion complex comprises a selenium compound, an iodine compound, and a silicon compound; selenium ions, iodine ions, and silicon ions in the selenium compound, the iodine compound, and the silicon compound have a mass ratio of 1.14-4.67:18.66-25:1.14-4.67; the selenium compound comprises sodium selenate; the iodine compound comprises potassium iodide; and the silicon compound comprises sodium metasilicate.

10. The method of claim 6, wherein the alkaline soluble rare earth salt comprises digermanate and lanthanum carbonate; the digermanate and the lanthanum carbonate have a mass ratio of 0.87-3.8:0.13-5.7.

11. The method of claim 6, wherein the composite gel comprises sodium alginate and nano silver; the sodium alginate and the nano silver have a mass ratio of 0.48-3:0.02-1.

12. An antiviral preparation, comprising the alkaline mineral composition of claim 1.

13. The antiviral preparation of claim 12, wherein the metal ion complex comprises a sodium compound, a potassium compound, a zinc compound, and an iron compound; sodium ions, potassium ions, zinc ions, and iron ions in the sodium compound, the potassium compound, the zinc compound, and the iron compound have a mass ratio of 16.48-19.02:41.21-43.18:0.23-0.25:2.06-7.93; the sodium compound comprises sodium chloride, sodium carbonate, sodium selenate, and sodium metasilicate; the potassium compound comprises potassium chloride, potassium iodide, and potassium carbonate; the zinc compound comprises zinc gluconate; and the iron compound comprises ferrous sulfate, ferrous lactate, and ferrous gluconate.

14. The antiviral preparation of claim 12, wherein the mineral ion complex comprises a selenium compound, an iodine compound, and a silicon compound; selenium ions, iodine ions, and silicon ions in the selenium compound, the iodine compound, and the silicon compound have a mass ratio of 1.14-4.67:18.66-25:1.14-4.67; the selenium compound comprises sodium selenate; the iodine compound comprises potassium iodide; and the silicon compound comprises sodium metasilicate.

15. A method for preventing and/or treating livestock infection caused by a virus, comprising administering to a subject in need thereof the alkaline mineral composition of claim 1.

16. The method of claim 15, wherein the virus comprises one or more of a coronavirus, a circovirus, a rotavirus, an arterivirus, and a herpesvirus.

17. The method of claim 15, wherein the metal ion complex comprises a sodium compound, a potassium compound, a zinc compound, and an iron compound; sodium ions, potassium ions, zinc ions, and iron ions in the sodium compound, the potassium compound, the zinc compound, and the iron compound have a mass ratio of 16.48-19.02:41.21-43.18:0.23-0.25:2.06-7.93; the sodium compound comprises sodium chloride, sodium carbonate, sodium selenate, and sodium metasilicate; the potassium compound comprises potassium chloride, potassium iodide, and potassium carbonate; the zinc compound comprises zinc gluconate; and the iron compound comprises ferrous sulfate, ferrous lactate, and ferrous gluconate.

18. The method of claim 15, wherein the mineral ion complex comprises a selenium compound, an iodine compound, and a silicon compound; selenium ions, iodine ions, and silicon ions in the selenium compound, the iodine compound, and the silicon compound have a mass ratio of 1.14-4.67:18.66-25:1.14-4.67; the selenium compound comprises sodium selenate; the iodine compound comprises potassium iodide; and the silicon compound comprises sodium metasilicate.

19. The method of claim 15, wherein the alkaline soluble rare earth salt comprises digermanate and lanthanum carbonate; the digermanate and the lanthanum carbonate have a mass ratio of 0.87-3.8:0.13-5.7.

20. The method of claim 15, wherein the composite gel comprises sodium alginate and nano silver; the sodium alginate and the nano silver have a mass ratio of 0.48-3:0.02-1.
